(19) United States
(12) Patent Application Publication
(10) Pub. No.: US 2003/0040753 A1
(43) Pub. Date: Feb. 27, 2003

(54) Cranial guide device and methods

(76) Inventors: Wolfgang Daum, Schwerin (DE); Axel Winkel, Zapel Hof (DE)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/226,364

(22) Filed: Aug. 22, 2002

Related U.S. Application Data
(63) Continuation of application No. 09/100,056, filed on Jun. 19, 1998, now abandoned.

(30) Foreign Application Priority Data
Jun. 19, 1997 (DE) 197 26 141.8-35

(51) Int. Cl. A61F 2/00

(52) U.S. Cl. 606/96

(57) ABSTRACT

The present invention is directed to devices and methods for accurately performing minimally invasive intracranial medical procedures. The invention provides a cranial guide device for guiding medical instruments to a selected site within the cranium of a patient as well as methods for using the devices of the invention.

### FIELD OF THE INVENTION

The invention is directed to performing an intracranial medical procedure. Specifically, the invention provides devices and methods for inserting and guiding medical instruments used to perform intracranial medical procedures.

### BACKGROUND OF THE INVENTION

Presently, surgical removal of brain tumors can require removal of large areas of the patient's skull. The removal of the skull results in an extended healing process as well as the frequent need for subsequent surgeries. Hence, often times in a modern neurosurgery, it is desirable to access the brain through the smallest opening size possible for taking a biopsy, administering a treatment, removing tumors, etc. In addition, the demand for precision during an intracranial medical procedure often makes it advantageous to perform certain intracranial procedures under such imaging systems as, for example, magnetic resonance tomography.

While stereotactic devices for performing intracranial procedures are available, these devices are frequently complicated to use and rely on a reference point relative to the operating table rather than the patient's skull. Thus, movement of the patient's head relative to the operating table can be disadvantageous to maintaining stereotactic alignment. Complicated devices for alignment relative to the head have been developed. However, many of these devices are large, cumbersome to use and expensive.

Accordingly, there is a continuing need for instrumentation and methods for performing neurosurgical procedures precisely under minimally invasive conditions.

### SUMMARY OF THE INVENTION

The present invention claims priority to German patent application 197 26 141.8-35, the entire disclosure of which is incorporated herein by reference.

The invention is directed to devices and methods for performing an intracranial medical procedure. In general, a device of the invention includes a cranial guide device including a cranial trocar and a guiding cannula. The cranial trocar includes an anchoring arrangement for anchoring the cranial guide device to the patient's cranium and a trocar lumen passing through the cranial trocar. The guiding cannula is insertable within the trocar lumen and includes a lumen for passing a medical instrument into the patient's cranium.

In some embodiments, the anchoring device includes threads for threadedly anchoring the cranial guide device to the patient's cranium. The threads can be self tapping threads.

In one embodiment, the guiding cannula can be part of a pivotally rotatable socket joint that mounts within the lumen of the cranial trocar. The rotational position of the guiding cannula can be fixed by a compression clamp surrounding the socket joint.

In some preferred embodiments, the cranial guide device can be manufactured from a material that can be used under magnetic resonance imaging without causing an interfering artifact on an image created by the MRI.

The cranial guide device provides for passage of a medical instrument into a patient's cranium. In one embodiment, the medical instrument is a deflectable needle having a pre-bent distal end. According to this embodiment, the medical instrument can be a single deflectable needle or a plurality of telescoping deflectable needles. In another embodiment, a multilumen working tube can be passed through the guide tube lumen or a deflectable needle for providing multiple operating channels through the cranial guide device.

Many other embodiments of a cranial guide device are disclosed. The invention also provides methods for using a cranial guide device to perform an intracranial medical procedure.

### BRIEF DESCRIPTION OF THE DRAWINGS

FIG. 1a is a top perspective view of one embodiment of a cranial guide device according to the invention;

FIG. 1b is a side perspective view of the embodiment of the cranial guide device of FIG. 1a;

FIG. 2 is a cross-section view through the embodiment of the cranial guide device of FIGS. 1a and 1b;

FIG. 3 is a side view of the embodiment of the FIGS. 1 and 2 including a guiding cannula cranial guide device;

FIG. 4 is a top perspective view of the embodiment of the cranial guide device shown in FIG. 3;

FIG. 5a is a cross section view of an embodiment of a cranial trocar according to the invention;

FIG. 5b is a top perspective view of an embodiment of a cranial guide device according to the invention;

FIGS. 6a-6e diagrammatically illustrate a method according to the invention;

FIGS. 7a and 7b illustrate an embodiment of an insertion tool according to the invention;

FIGS. 8a and 8b illustrate a cylindrical surgical field provided by a cranial guide device according to the invention;

FIGS. 9a-9e illustrate various embodiments of a working cannula according to the invention;

FIG. 10 diagrammatically illustrates various diagnostic and therapeutic procedures which can be performed according to the invention; and

FIGS. 11a-11b illustrate another embodiment of a cranial guide device according to the invention, FIGS. 11b taken at line 11b-11b of FIG. 11a.

### DETAILED DESCRIPTION OF THE PREFERRED EMBODIMENT

The present invention is directed to devices and methods for performing intracranial medical procedures. As used herein, the term “intracranial” refers to procedures performed within the bones of the cranium or skull. Typically, these are procedures performed on the brain, dura mater, arachnoid, pia mater, etc. Also, in this context, the term “medical procedure” includes any medical, surgical, therapeutic or diagnostic procedure that can be performed within the cranium of a patient.

In one embodiment, the invention provides a cranial guide device for accessing the intracranial region of a patient. Preferably, the cranial guide device is anchored to a skull bone of the patient and provides a passage for inserting a medical instrument into the intracranial regions of the patient from the exterior of the cranium. Anchoring the cranial guide device to the patient's cranium provides a fixed reference point for precise positioning of medical instruments which are passed through the cranial guide device to perform the intracranial medical procedure. As used herein, a “medical instrument” includes any instrument which may be used by a physician in performing a procedure in the intracranial region of a patient that can be inserted into the intracranial region through a cranial guide device of the invention. Such medical instruments include, for example, needles, catheters, endoscopes, fiber optic instruments, laser instruments, coagulation instruments, forceps, retractors, etc.

The cranial guide devices can be manufactured from materials known in the art, such as stainless steel, using known methods of manufacture. In some embodiments, the cranial guide device, or components thereof, can be manufactured from materials that cause low, or no, interfering artifact on diagnostic images, such as those taken by magnetic resonance imaging (MRI). As used herein, “interfering artifact” means distortion, shadow, or other anomaly on the diagnostic image that is caused by a device of the invention and which substantially interferes with proper interpretation of the diagnostic image by the physician.

### DETAILED DESCRIPTION OF THE ILLUSTRATED EMBODIMENTS

Referring now to the several drawing figures in which identical elements are numbered identically throughout, a description of the illustrated embodiments of the present invention will be provided.

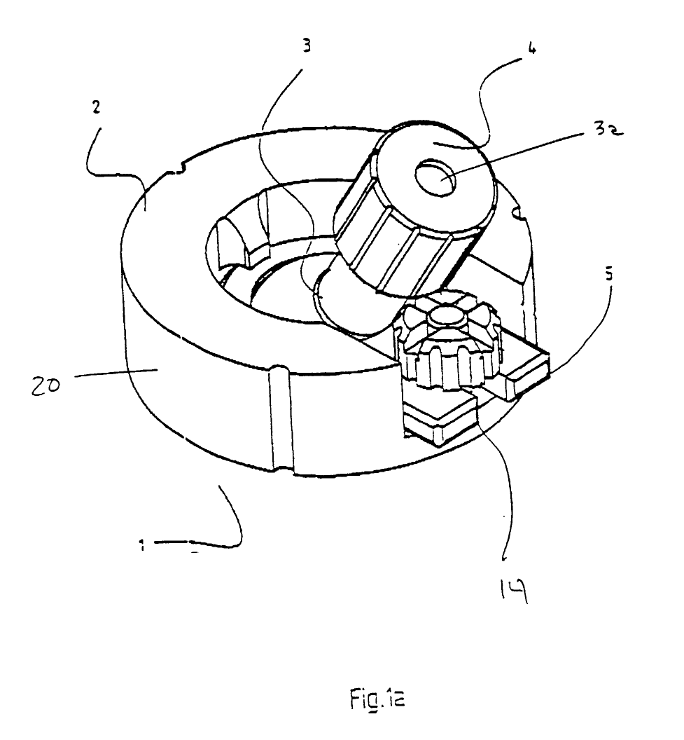

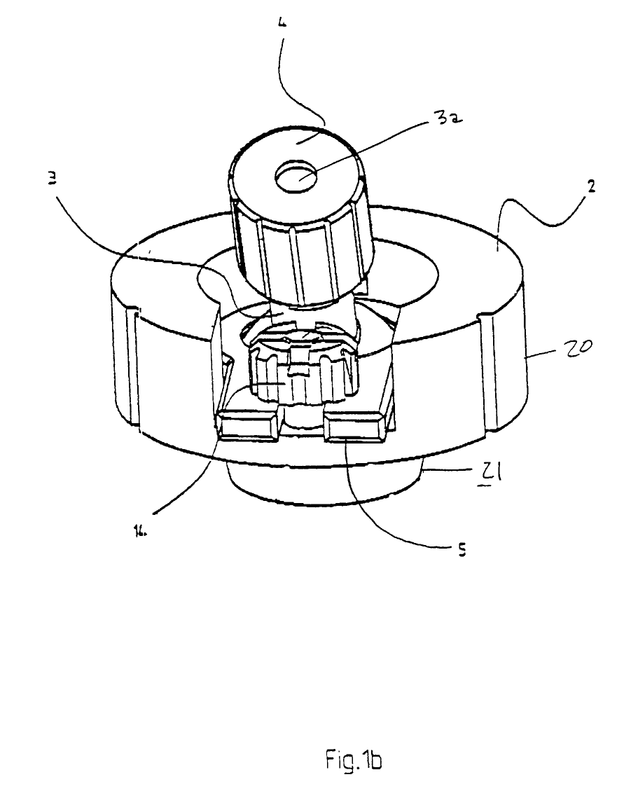

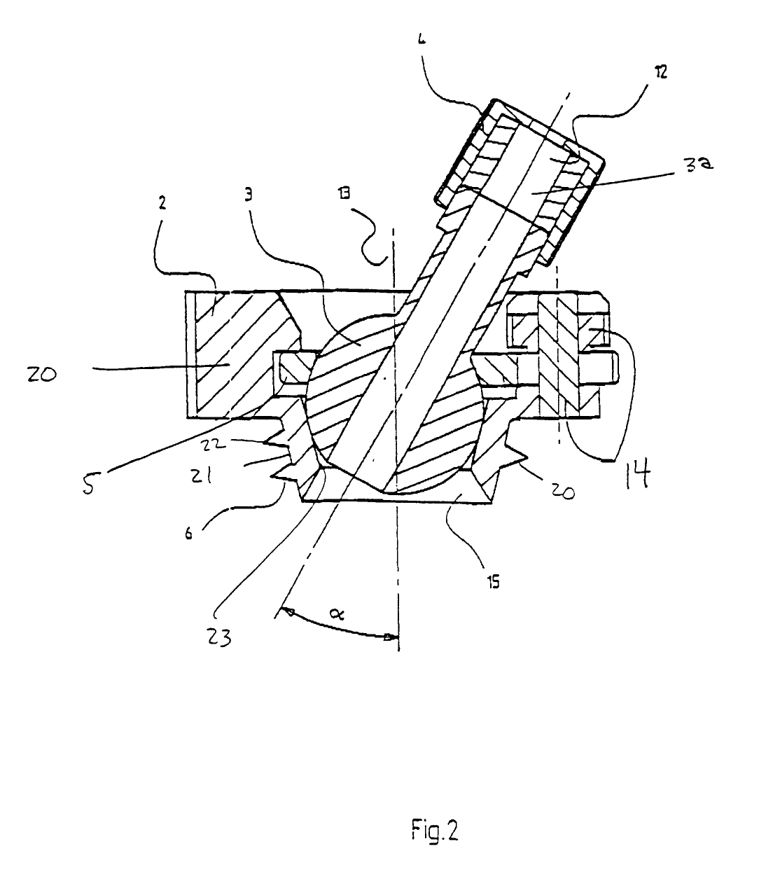

FIGS. 1-4 illustrate a first embodiment of a cranial guide device (neurotrocar device). In this embodiment, cranial guide device 1 includes a cranial trocar 20 and a guiding cannula (needle holder) 3. The cranial trocar 20 includes a fastening plate 2 and an anchoring arrangement 21, such as threads 22. In some embodiments, threads 22 can be self tapping such as, screw-tap 6. Also, in some embodiments, fastening plate 2 can function as a positive stop when inserting cranial guide device 1 into the patient's skull. In an alternative embodiment, anchoring arrangement 21 can be a quarter-turn fastener (bayonet), spreader, self-locking fixing device or a clamping joint.

The guiding cannula 3 provides an insertion tube through which a medical instrument can be passed. In a preferred embodiment, the lumen 3a of the guiding cannula 3 is circular in cross section. However, other geometrical configurations of the cross section of the lumen can be used.

Cranial guide device 1 includes a socket joint 15 which provides for rotation of the guiding cannula 3 relative to the fastening plate 2. The orientation of the guiding cannula 3 can be fixed by tightening clamp 5 around socket joint 15 by rotating knurled screw 14. An instrument passed through lumen 3a of guiding cannula 3 can be fixed in position by rotation of fixing device 4. Fixing device 4 can be, for example, a Touhy Borst valve, trumpet valve or rubber gasket.

In a preferred embodiment, the cranial guide device is manufactured from a material which can be used during magnetic resonance imaging (MRI). In a preferred embodiment, the cranial guide device 1 will create only minimal or no interfering artifact on an MRI image. Suitable materials which cause minimal or no interfering artifact include, for example, titanium alloys as described in German Patent Application DE 195 31 117.5-35 and co-pending U.S. application Ser. No. 08/639,215, the entire disclosures of which are incorporated herein by reference. In addition, ceramic, synthetic materials such as plastics (e.g., polyetheretherketene, PEEK), or chrome plated brass or aluminum alloys can be used.

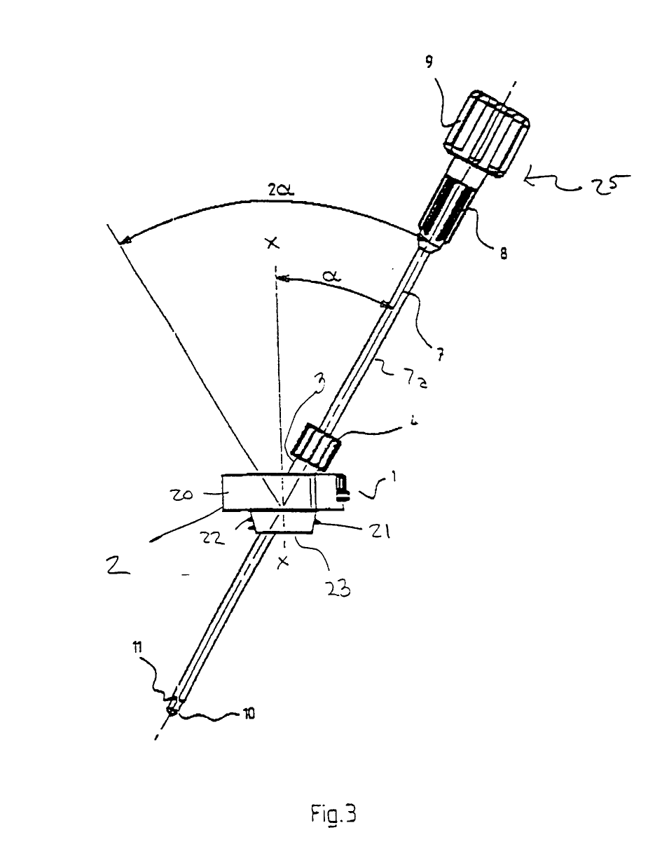

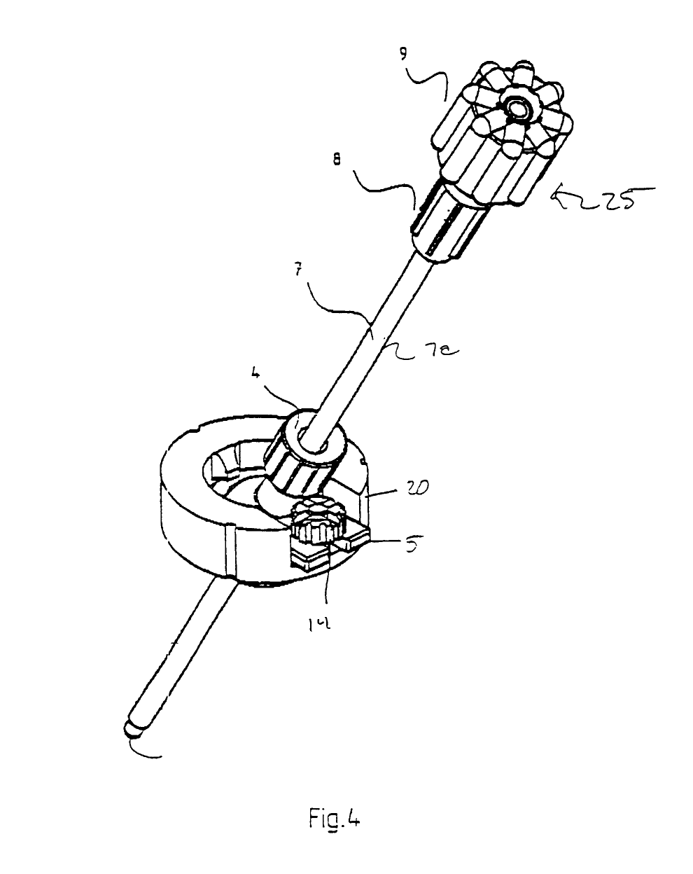

FIGS. 3 and 4 illustrate a medical instrument 7, such as a needle 7a, passed into lumen 3a of guiding cannula 3. Guiding cannula 3 includes a fixation device 4 which can fix the position of needle 7a within fixing device 4 such as fixing the insertion depth of needle 7a. The needle 7a can be a neuronal aspiration needle having a circular (rounded) ground tip 11. A guidewire 10 is illustrated passing through needle 7a. At the proximal end 25, the needle can include a connecting arrangement 8, such as a Leur Lock 8 and a grip 9 for operating the guidewire. As illustrated in FIG. 3, the guiding cannula 3 can be rotated relative to the fastening plate 2, or relative to the longitudinal axis X-X of the lumen 23 of cranial trocar 20, around angle α.

In use, a small perforation is made into the cranium and the cranial guide device is secured in the perforation by anchoring arrangement 21. Once inserted into the cranium, the cranial guide device 1 can be used as a guide for accurately positioning other medical instruments which can be passed through guiding cannula 3. If desired, the cranial guide device 1 can be sterilely sealed if the cranial guide device is to be left in the patient's cranium for a period of time, that is, for a period of time longer than that required for a single procedure.

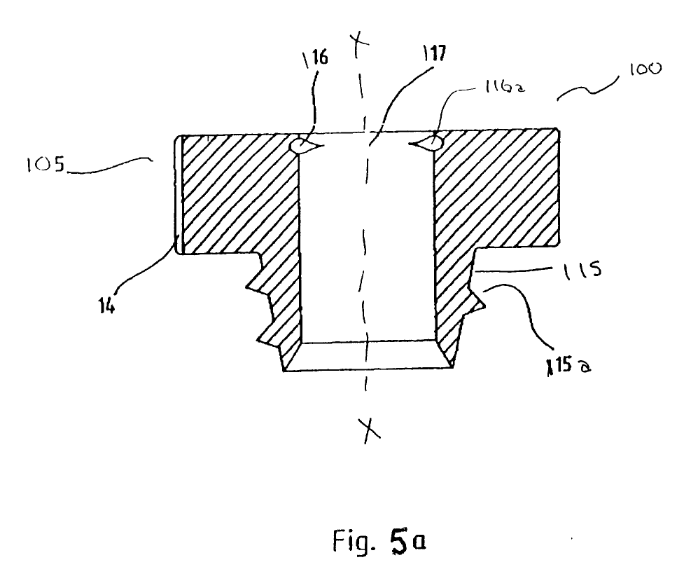

Referring now to FIGS. 5-11, alternative embodiments for a cranial guide device and methods of use will be described.

FIG. 5a is a cross section view of another embodiment of a cranial trocar 105 that does not include a pivotally rotatable guiding cannula as does the embodiment of FIGS. 1-4. Like the embodiment described above, the trocar lumen 117 of cranial trocar 105 has a longitudinal axis X-X. Cranial trocar 105 includes an anchoring arrangement 115 having self cutting threads 115a. A seal 116, such as gasket 116a, is present within lumen 117 to provide a seal between lumen 117 and the outer surface of an instrument passed through lumen 117.

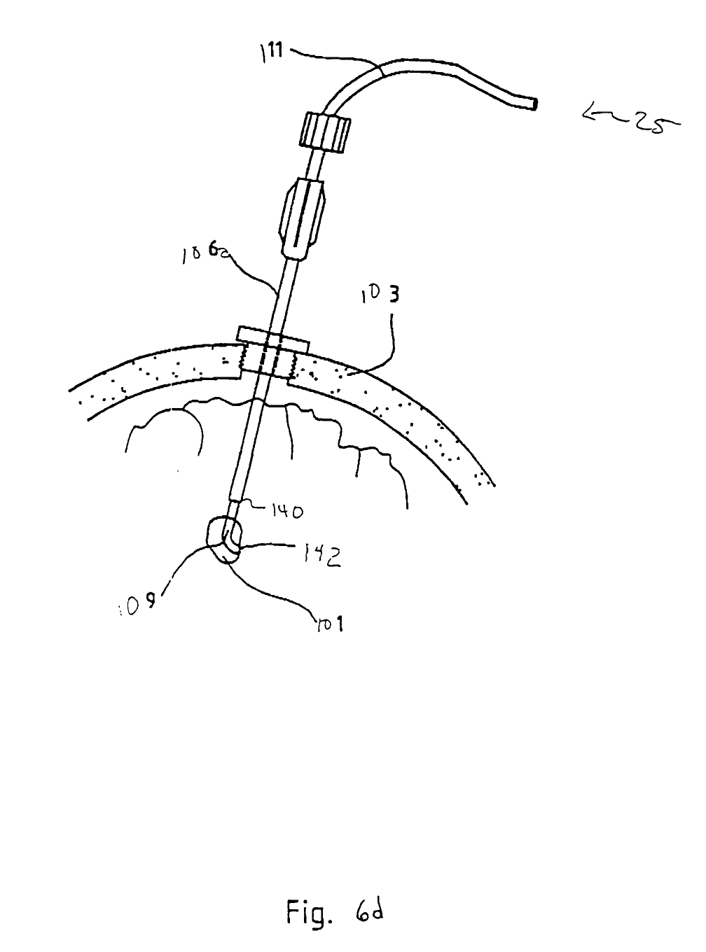

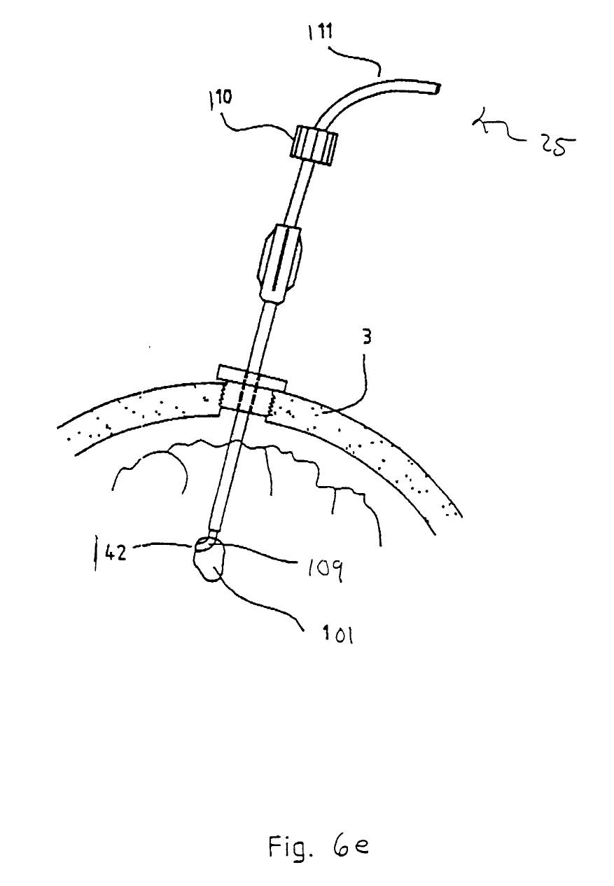

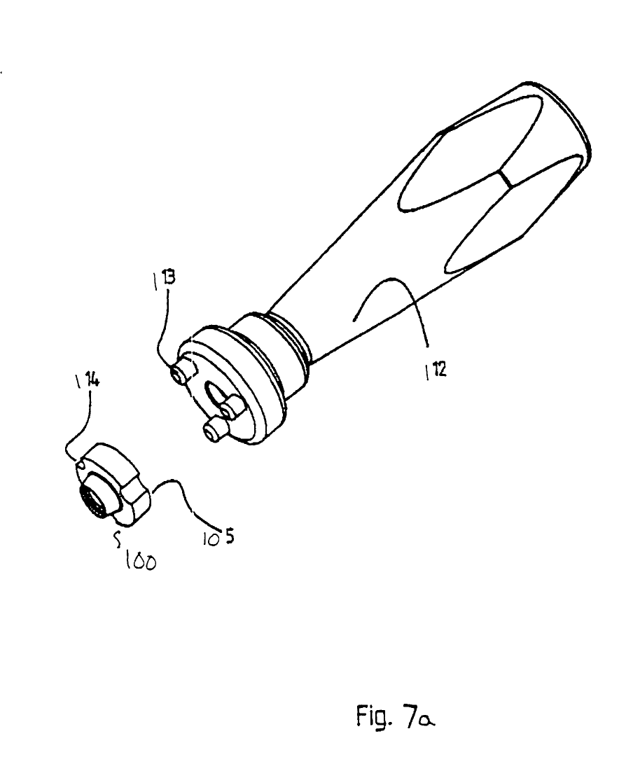

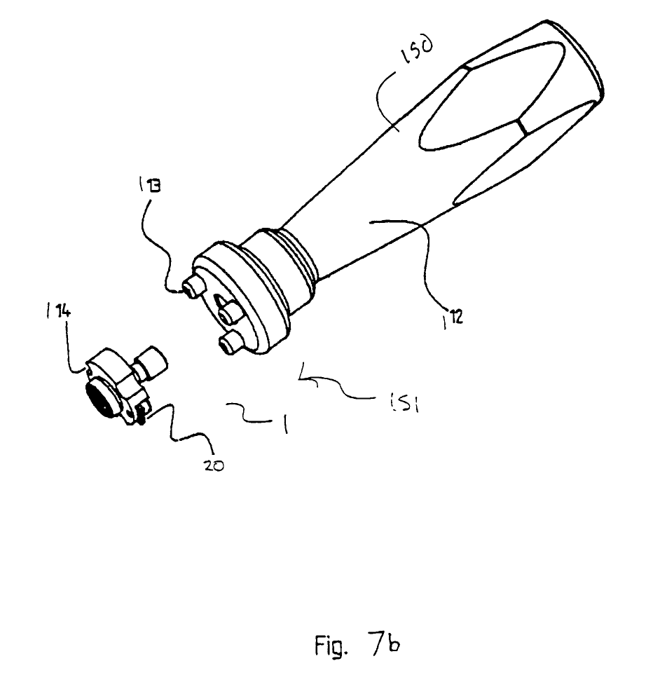

As illustrated in FIG. 5b, the exterior surface of cranial trocar 105 includes two or more indentations for complementary mating to components of an insertion tool 112 illustrated in FIGS. 7a and 7b. Insertion tool 112 includes a handle 150 for operation of the insertion tool 112 by an operator. At the distal end 151 of insertion tool 112, three protuberances 113 mate with indentations 114 of cranial trocar 105. The protuberances can be, for example, the head of bolts with a size appropriate for fitting within indentations 114. The complimentary fit of insertion tool 112 with cranial trocar 105 permits rotation of cranial trocar 105 for insertion into skull bone 103. FIG. 7b illustrates use of insertion tool 112 with an embodiment of cranial guide device 1.

In an alternative embodiment, the cranial trocar 105 can be fixed to the skull bone 103 with a clamping seal or a Velcro fastener.

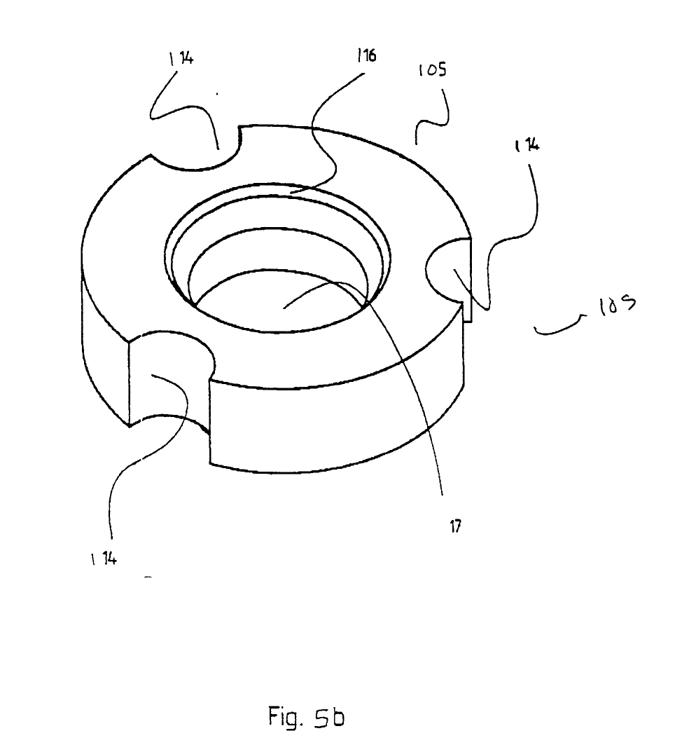

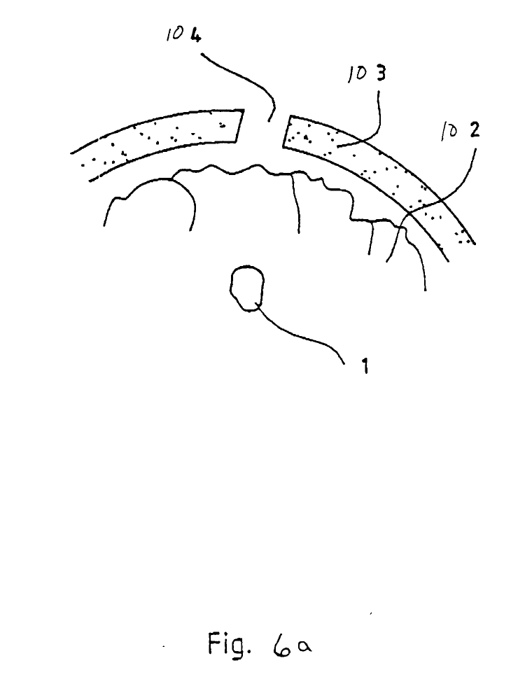

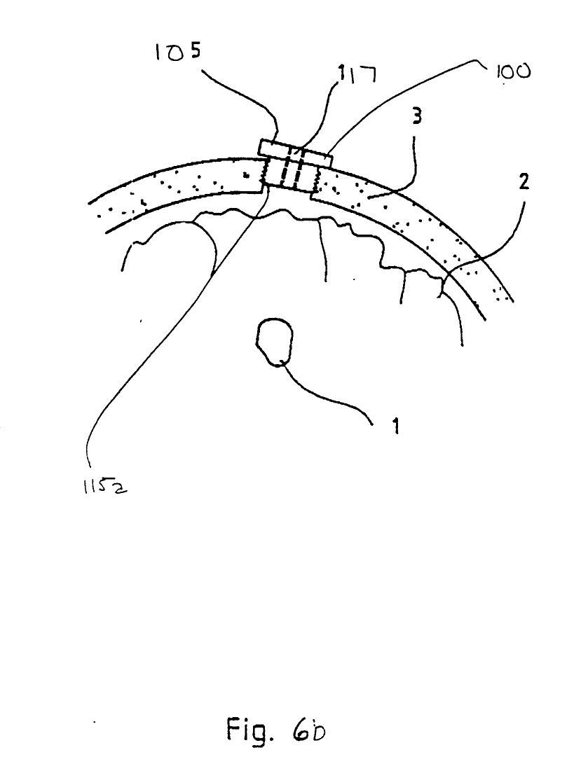

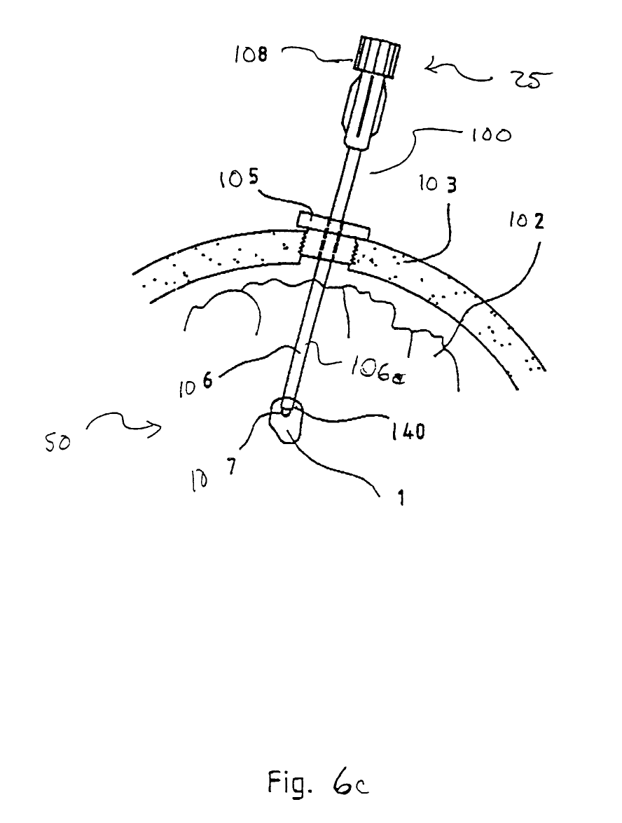

FIGS. 6a-6e illustrate a method of using the cranial guide device 100 (or cranial guide device 1) for therapy of a brain tumor. In FIG. 6a, a drill hole 104 is made through skull bone 103 to access brain tissue 102. A deep seated tumor 101 is diagrammatically illustrated deep to skull bone 103. FIG. 6b illustrates placement of a cranial trocar 105 of cranial guide device 100 having an anchoring arrangement comprising a self-cutting threads 115a. The trocar lumen (channel) 117 is illustrated by phantom lines.

Referring to FIG. 6c, once the cranial trocar 105 is screwed into the skull bone 103, a guiding cannula 106a, such as outer needle 106, including an inner mandarin 107 is passed through the cranial trocar 105 to the tumor 101. The proximal end 25 of guiding cannula 106a can include a connecting arrangement 108, such as a Leur or Leur Lock connection. The distal end 50 of guiding cannula 106a includes a rounded atraumatic distal tip 140. In some embodiments of the invention, the guiding cannula 106a can be passed to tumor 101 under MRI visualization without interfering artifact from the guiding cannula or other components.

Referring to FIG. 6d, once the distal tip 140 of guiding cannula 106a is positioned at tumor 101, the mandarin 107 can be pulled proximally out of guiding cannula 106a and replaced by a medical instrument. In the illustrated embodiment, the medical instrument is a deflectable needle 109 having a distal end pre-bent to a predetermined angle. The deflectable needle 109 can be designed and operated as described in, for example, German application DE 42 23 897.8 and copending U.S. application Ser. No. 08/552,143, the disclosures of which are incorporated herein by reference. Deflectable needle 109 can provide for aspiration of a material from the intracranial region or infusion of a medicament into the intracranial region. As used herein, a “medicament” includes any exogenous or endogenous substance that provides a diagnostic, therapeutic or palliative effect to the patient. This includes, for example, antibiotics, chemotherapy, electrolyte solutions, analgesic agents, contrast agents, etc.

Preferably, guiding cannula 106a is inflexible such that when deflectable needle 109 is passed through guiding cannula 106a, deflectable needle 109 is straight within the lumen of guiding cannula 106a. Deflectable needle 109 is preferably prepared from any material capable of maintaining the pre-bent shape and capable of returning to the pre-bent shape after being forceably straightened by insertion into the lumen of guiding cannula 106a. Suitable materials for a deflectable needle include plastic, rubber, elastic, super elastic (pseudo elastic), alloys, and other materials having shape memory. One preferred material is nickel-titanium which provides for elasticity of the needle but still maintains the rigid properties of a metal. Materials suitable for a guiding cannula 106a include, for example, alloys, including alloys of titanium, rigid plastics (e.g., polyetheretherketene, PEEK), ceramics, etc.

By comparing the distal tip 142 of deflectable needle 109 in FIGS. 6d and 6e, it will be appreciated that various regions of tumor 101 can be aspirated with a single deflectable needle 109 by passing deflectable needle 109 through guiding cannula 106a to a particular region, then withdrawing deflectable needle 109 proximally into guiding cannula 106a and rotating deflectable needle 109 to a new position before advancing it distally into tumor 101 in a new location. Additional deflectable needles can be passed through the lumen of a first deflectable needle, such as deflectable needle 109, to extend the accessible working space of cannula 109. Such a telescoping system of deflectable needles is discussed further in, for example, co-pending U.S. patent application Ser. No. 08/552,143. A tube 111 can be connected to the proximal end of deflectable needle 109 by a connector 110 through which the tumor or other material can be aspirated from the cranial region.

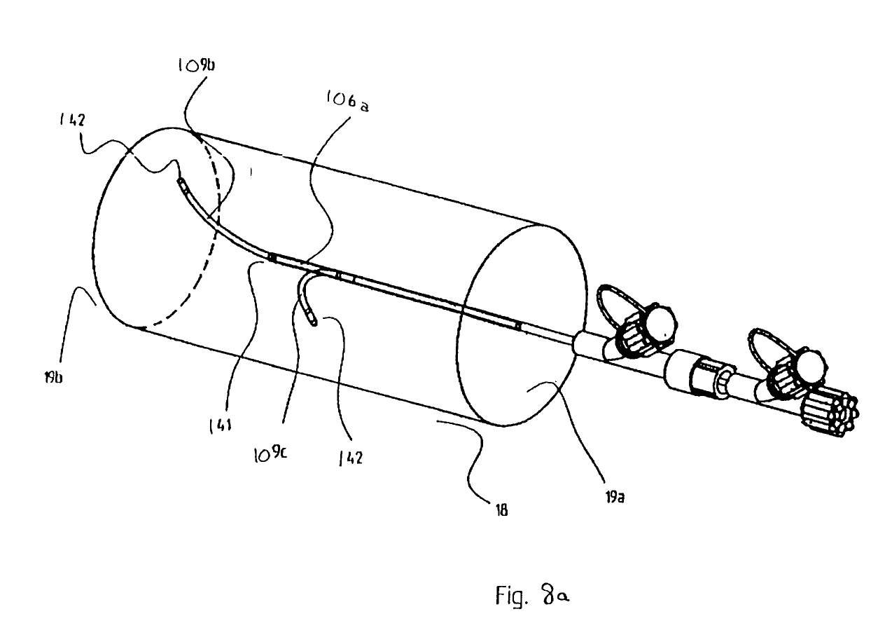

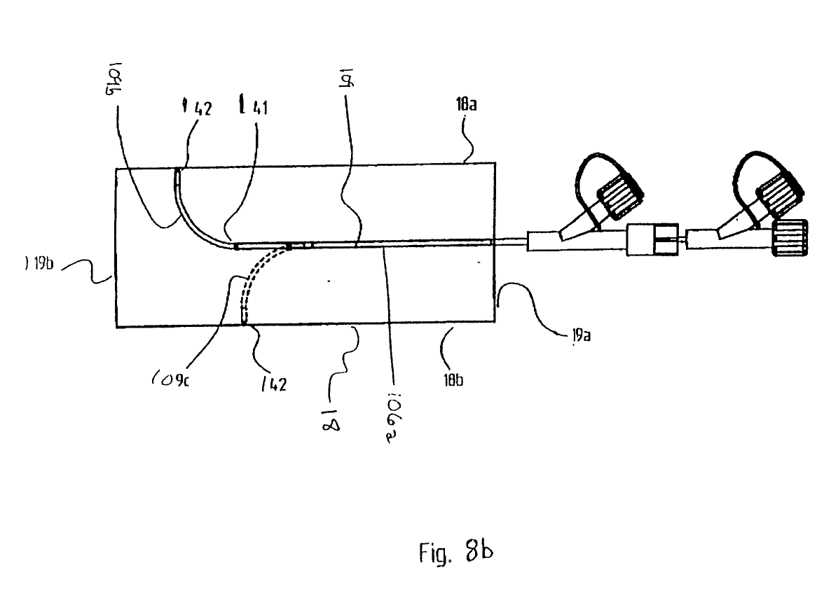

FIGS. 8a and 8b illustrate a cylindrical surgical field which is available to a surgeon using an embodiment of a cranial guide device and deflectable needle 109 as disclosed herein. According to this embodiment, a guiding cannula 106a passed through the lumen of cranial trocar 105 (not illustrated in FIGS. 8a and 8b) can access an intracranial region approximately defined by a cylinder 18. That is, the distal tip 142 of deflectable needle 109 can be placed at any point selected within cylindrical surgical field 18. Thus, once the distal tip 141 of guiding cannula 106a is passed into the cranium, advancing the distal tip 142 of deflectable needle 109 beyond the distal tip 141 of guiding cannula 106a places the distal tip 142 of deflectable needle 109 at a certain location. Retracting deflectable needle 109 proximally within guiding cannula 106a and rotating the deflectable needle 109 will reposition distal tip 142 at a new location when the distal tip 142 of deflectable needle 109 is readvanced beyond the distal tip 141 of guiding cannula 106a.

FIG. 8b illustrates two needle positions, 109b and 109c. However, by advancing and retracting guiding cannula 106a distally and proximally and retracting, rotating and advancing the distal tip 142 of deflectable needle 109, all regions within the bases 19a and 19b of cylinder 18 can be accessed. Thus, a complete inner cylinder volume with the bases 19a and 19b and outer surface 18 can be aspirated.

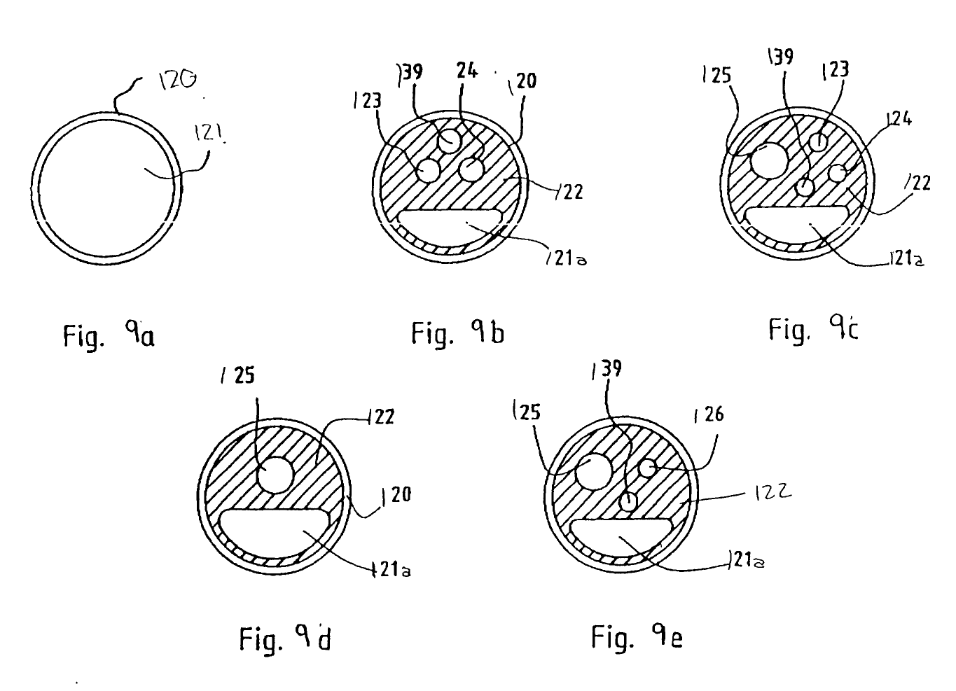

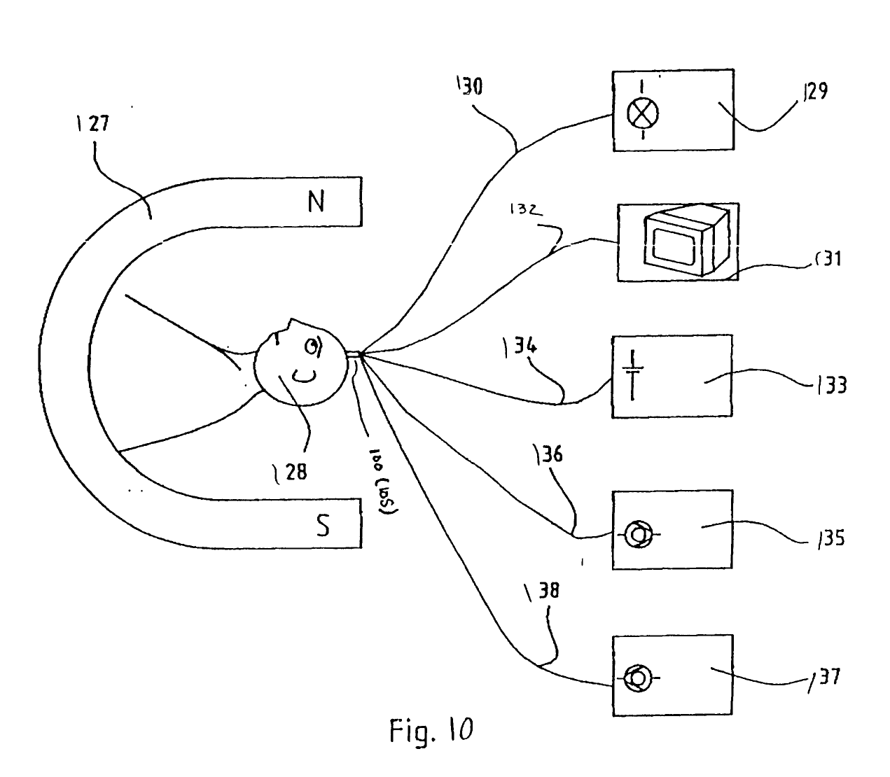

FIGS. 9a-9e, illustrate transverse cross section views of instruments that can be passed through a guiding cannula 3 (or through a deflectable needle 109). For purposes herein, 120 refers to a guiding cannula or deflectable needle (i.e., working cannula) and 121 refers to a lumen passing therethrough. FIG. 10 diagrammatically illustrates placement of a patient 128 within an MRI unit including a cranial guide device 100 (105) of the invention. It will be appreciated that for optimal results, all materials passed through the cranial guide device 105 for use under MRI imaging be manufactured from materials that are suitable for use under MRI conditions. That is, preferably the materials neither deflect the magnetic waves nor cause artifact or other image distortion on the MRI image. Suitable materials can be, for example, Ti, Ti6A1-4V, Ti-6A1-6V2SN, Ti-3A1-2.5V, NiTi, synthetic materials such as plastics, ceramics, etc. For medical operations not performed within the field of an MRI, the material of the instruments passed into a cranial guide device of the invention can be any medically approved material including stainless steel, high grade steel, ceramics or synthetic materials such as polypropylene, polystyrene, polyethylene, polymethylmethacrylate (PMMA), etc.

Referring now to FIG. 9a, the inner dimensions of working cannula 120 are limited only by patient tolerance. Generally, the lumen 121 diameter can be about 1 mm to 10 mm, typically about 2 mm to 6 mm. The embodiment of FIG. 9a includes a single lumen 121 for aspirating a material from the intracranial region or passing a material, such as a drug or flushing solution, into the intracranial region. However, multilumen instruments 122 can also be passed through working cannula 120. As shown in FIG. 9b, a multilumen inner body 122 can include an aspiration lumen 121a, a flushing lumen 139, and one or more lumens 123 and 124 for passing electrodes of a coagulation unit. The electrode (not illustrated) could be passed through either of lumens 123 or 124 to stanch cerebral hemorrhages bipolarly.

In FIG. 9c, the multilumen inner body 122 further includes a lumen 125 for insertion of an endoscope for visualization of the intracranial region accessed. In FIG. 9d, another embodiment of a multilumen inner body 122 is illustrated having an aspiration lumen 121a and an endoscope lumen 125 for passing an endoscope. FIG. 9e illustrates a multilumen inner body 122 having a monopolar coagulation lumen 126 for a monopolar electrode wire for monopolar coagulation of a cerebral hemorrhage. A second pole for the monopolar coagulation unit would be passed through a patient's external skin connection. The multilumen inner body 122 of FIG. 9e also illustrates lumens 121a and 139 and endoscope lumen 125. It will be appreciated that other permutations of lumen arrangements for multilumen working bodies are within the scope of the invention.

FIG. 10 illustrates the various diagnostic and therapeutic capabilities which can be performed through a cranial guide device of the invention. Examples of FIG. 10 include a lighting unit 129 using a light feed 130 such as a glass fiber cable, to pass light into the surgical field. The light feed 130 can pass through a separate lumen not illustrated in FIGS. 9a-9e or through endoscope lumen 126. In addition, visualization can also be provided through a camera mounted to an endoscope and viewed by a monitor 131, with optical transmission through cable 132. A coagulation unit 133 passing current via cable 134 is illustrated for stanching cerebral hemorrhage. An aspiration lumen 135 can aspirate a tumor via aspiration channel 136 which could be a hollow tube. In addition, flushing medium such as water or physiological saline solution can be passed into the operation area with a flushing pump 137 through a feeding channel 138.

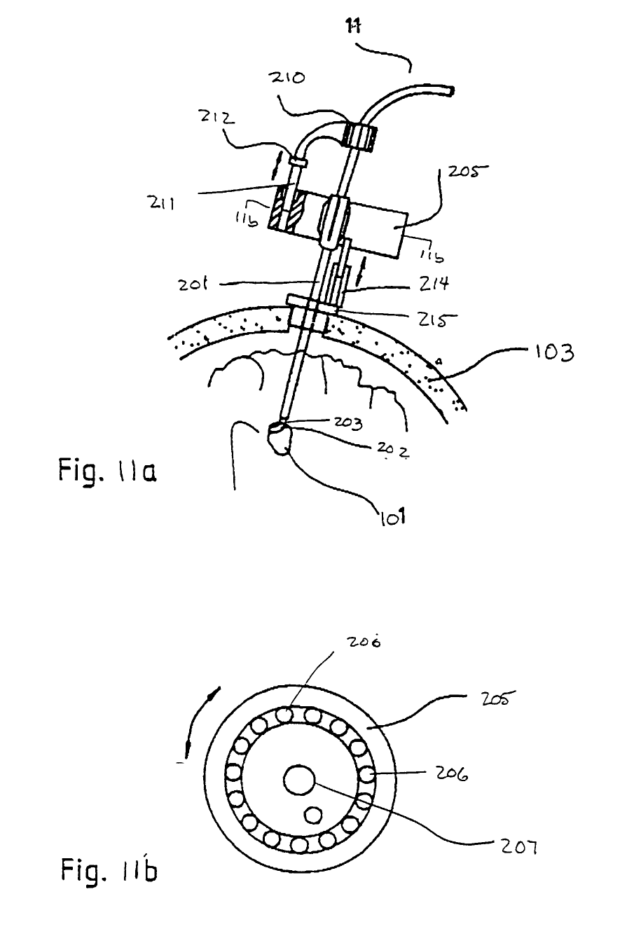

FIGS. 11a and 11b illustrate an embodiment of a cranial guide device for accessing regions within a patient's cranium with greater control for safety. According to this embodiment, the diameter of a cylindrical working field is determined by a defined travel distance of the guiding cannula 201 and working needle 202, such as deflectable needle 203. Guiding cannula 201 includes a guiding handle 205 having multiple channels 206. Working handle 210 of working needle 202 includes guide pin 211 and guide pin stop 212. Guide pin 211 is configured for sliding fit into the channels 206 up to guide pin stop 212. In addition, an axial travel limiter such as adjustable spacer 214 controls axial travel of guiding cannula 201. Adjustable spacer 214 is positioned between fastening plate 215 and guiding handle 205. The depth of penetration of guiding cannula 201 (i.e., axial travel) can be incrementally adjusted by adjustable spacer 214. Thus, the axial travel of working needle 202 is controlled by the axial travel adjustable spacer 214 and the axial travel of guide pin 211. Axial travel of guiding cannula 201 and working needle 202 determine the length of the cylindrical field. The angular position of working needle 202 is controlled by proximal retraction of working handle 210 until guide pin 211 is clear of channels 206 and rotating working handle 210 radially. Working needle 202 can be incrementally advanced radially to locations where guide pine 211 aligns with a channel 206. It will be appreciated that although FIG. 11b illustrates channels 206 as having a circular cross section any shape is sufficient that permits interdigitation between guide pin 211 and channels 206 to perform the described function.

From the foregoing detailed description of the present invention it has been shown how the objects of the invention have been obtained in a preferred manner. However, modifications and equivalence of the disclosed concepts such as those which would occur to one of ordinary skill in the art are intended to be included within the scope of the claims and their equivalents.

We claim:

1. A cranial guide device for performing an intracranial medical procedure, the cranial guide device comprising:
a cranial trocar comprising:
(i) an anchoring arrangement for anchoring the cranial guide device to a patient's cranium;
(ii) a trocar lumen having a longitudinal axis, the trocar lumen providing for passage of a medical instrument through the cranial trocar; and
a guiding cannula having a lumen, the guiding cannula insertable within the trocar lumen and providing for passage of the medical instrument into the patient's cranium.

2. A cranial guide device according to claim 1 wherein the cranial trocar further comprises a fastening plate surrounding the trocar lumen.

3. A cranial guide device according to claim 1 wherein the anchoring arrangement comprises threads for threadedly anchoring the cranial guide device to the patient's cranium.

4. A cranial guide device according to claim 1 wherein the guiding cannula can be pivotally rotated to a selected angle relative to the longitudinal axis of the trocar lumen.

5. A cranial guide device according to claim 4 wherein the guiding cannula is pivotally rotatable by a socket joint.

6. A cranial guide device according to claim 4 wherein the guiding cannula can be fixed at the selected angle.

7. A cranial guide device according to claim 6 wherein the guiding cannula is fixed selected angle by a compression clamp.

8. A cranial guide device according to claim 1 wherein the cranial guide device can maintain a sterile access port to the patient's cranium for a period of time.

9. A cranial guide device according to claim 4 wherein the selected angle of the guiding cannula can be changed and subsequently returned back to the selected angle.

10. A cranial guide device according to claim 1 wherein the cranial guide device is of a material that can be used under magnetic resonance imaging (MRI) without causing an interfering artifact on an image created by the MRI.

11. A cranial guide device according to claim 4 wherein a medical instrument passed through the guiding cannula can be fixed at a selected position within the guiding cannula.

12. A cranial guide device according to claim 1 wherein the medical instrument is a deflectable needle that can be passed through the guiding cannula.

13. A cranial guide device according to claim 12 wherein the deflectable needle is pre-bent at a distal end.

14. A cranial guide device according to claim 1 wherein the medical instrument is a plurality of telescoping deflectable needles.

15. A cranial guide device according to claim 1 further comprising an insertion tool for inserting the cranial guide device into the patient's cranium.

16. A cranial guide device according to claim 1 wherein the medical instrument is a multilumen working tube comprising at least two lumens.

17. A cranial guide device according to claim 16 wherein the multilumen working tube includes an endoscopic channel for guiding an endoscope into the patient's cranium.

18. A cranial guide device according to claim 16 wherein at least one lumen of the multilumen working tube is an aspiration lumen.

19. A cranial guide device according to claim 16 wherein at least one lumen of the multilumen working tube provides for passage of an electrode of a coagulation unit.

20. A cranial guide device according to claim 16 wherein at least one lumen of the multilumen working tube is a flushing channel.

21. A cranial guide device according to claim 1 wherein the cranial guide device comprises a material selected from the group consisting of nickel-titanium alloy, Ti (grade 2), Ti-6A1-4V (grade 5), Ti-3A1-2.5V (grade 9), Ti-6A1-6V-25n, ceramic or other material which does not form an artifact on a magnetic resonance imaging (MRI) image.

22. A cranial guide device according to claim 12 wherein the deflectable needle comprises a material selected from the group consisting of an elastic or pseudoelastic material.

23. A cranial guide device according to claim 1 wherein the guiding cannula has an atraumatic distal end.

24. A cranial guide device according to claim 12 wherein the deflectable working needle includes a beveled distal end.

25. A cranial guide device according to claim 3 wherein the threads are self tapping threads.

26. A cranial guide device according to claim 1 wherein the anchoring arrangement is a selected one of a quarter-turn fastener or a clamping joint.

27. A cranial guide device according to claim 16 wherein the multilumen working tube can pass through a deflectable needle.

28. A cranial guide device according to claim 1 wherein the cranial guide device comprises stainless steel.

29. A cranial guide device according to claim 2 further comprising a control arrangement for controlling the working region for performing the intracranial medical procedure, the control arrangement comprising:
a guide handle mounted at a proximal end of the guiding cannula, the guide handle having:
(i) a plurality of channels positioned radially around the guiding cannula lumen;
a deflectable needle insertable through the guiding cannula lumen, the deflectable needle having:
(i) a needle handle at a proximal end of the deflectable needle;
(ii) a guide pin extending distally from the needle handle and sized for complementary fit with at least one of the plurality of channels of the guiding handle;
(iii) a guide pin stop at a proximal end of the guide pin; and
an axial travel limiter mounted between the guiding handle and the fastening plate to control axial travel of the guiding cannula relative to the cranial trocar.

30. A method for performing an intracranial medical procedure on a patient, the method comprising:
anchoring a cranial guide device to the patient's cranium, the cranial guide device having:
(i) an anchoring arrangement for anchoring the cranial guide device to a patient's cranium;
(ii) a trocar lumen having a longitudinal axis and providing for passage of a medical instrument through the cranial trocar; and
passing a medical instrument through the cranial guide device into the patient's cranium;
performing the intracranial medical procedure through the cranial guide device.

31. A method according to claim 30 wherein the anchoring arrangement comprises threads external to the trocar lumen for anchoring the cranial guide device to the patient's cranium.

32. A method according to claim 30 wherein the cranial guide device further includes a guiding cannula insertable within the trocar lumen that provides for passage of the medical instrument through the cranial guide device.

33. A method according to claim 32 wherein a deflectable needle is passed through the guiding cannula to perform the intracranial medical procedure.

34. A method according to claim 33 wherein the intracranial medical procedure comprises removing a tumor from the patient's brain through the deflectable needle.

35. A method according to claim 33 wherein a medicament or gas is passed through the deflectable needle.

36. A method according to claim 30 wherein the medical instrument is a multilumen working tube comprising at least two lumens.

37. A method according to claim 28 wherein the deflectable needle provides for passing therethrough a selected one of a laser fiber, cryotherapy fiber, forceps or retractor.

38. A method according to claim 30 wherein the intracranial medical procedure is performed under magnetic resonance image visualization.
